(19) United States
(12) Patent Application Publication
(10) Pub. No.: US 2021/0244029 A1
(43) Pub. Date: Aug. 12, 2021

(54) ANTIMICROBIAL COMPOSITIONS

(71) Applicant: Aseptic Health, LLC, Nashville, TN (US)

(72) Inventors: Michael Politopoulos, Chattanooga, TN (US); Autumn N. Ryan, Nashville, TN (US); Gustave J. Scannapieco, Nolensville, TN (US)

(21) Appl. No.: 17/143,148

(22) Filed: Jan. 6, 2021

Related U.S. Application Data
(63) Continuation-in-part of application No. 16/894,618, filed on Jun. 5, 2020.
Application No. 16/894,618 is a continuation-in-part of application No. PCT/US2000/040577, filed on Jul. 1, 2020.
(60) Provisional application No. 63/009,863, filed on Apr. 14, 2020.
(60) Provisional application No. 62/925,997, filed on Oct. 25, 2019.
(60) Provisional application No. 62/869,112, filed on Jul. 1, 2019.

(51) Int. Cl. A01N 59/14; A01N 59/00; A01N 33/12; A01N 25/02; A01N 25/30; A01N 25/22; A61K 8/20; A61K 8/41; A61K 8/19; A61K 8/24; A61K 8/365; A61Q 17/00

(52) U.S. Cl. CPC A01N 59/14; A01N 59/00; A01N 33/12; A01N 25/02; A01N 25/30; A61Q 17/005; A61K 8/20; A61K 8/416; A61K 8/19; A61K 8/24; A61K 8/365; A01N 25/22

(57) ABSTRACT

The present disclosure generally describes an antimicrobial composition comprising: a water solution comprising a chlorite salt having a concentration ranging from about 2,000 parts per million to about 8,000 parts per million, and at least one quaternary ammonium salt having a concentration ranging from about 5,000 parts per million to about 10,000 parts per million. The present compositions are advantageously effective against a variety of bacteria, viruses, molds, and fungi, and may be used in a variety applications. Such applications include, without limitation, healthcare setting and equipment disinfection, food surface disinfection, agricultural disinfection, and personal hand care disinfection.

### CROSS-REFERENCES TO RELATED APPLICATIONS

This application is a continuation-in-part of U.S. application Ser. No. 16/894,618 filed on Jun. 5, 2020, which claims the benefit of U.S. Provisional Application Nos. 62/869,112 filed on Jul. 1, 2019, 62/925,997 filed on Oct. 25, 2019, and 63/009,863 filed on Apr. 14, 2020, each of which is incorporated by reference in its entirety. This application also is a continuation-in-part of international application no. PCT/US2020/040577 filed on Jul. 1, 2020, which is incorporate by reference in its entirety.

### TECHNICAL FIELD

The present disclosure generally relates to antimicrobial compositions and methods of using such antimicrobial compositions for killing harmful bacteria, viruses, funguses, molds, and the like.

### BACKGROUND ART

Clostridium difficile ATCC 43598 (C. difficile), Staphylococcus aureus (S. aureus), Escherichia coli (E. coli), Pseudomonas Aeruginosa (P. aeruginosa), Enterobacter aerogenes, and other harmful bacteria can be found in a variety of environments including, but not limited to, medical, industrial, residential, or food preparation environments. Exposure to these bacteria can cause illness, disease, and/or infection, particularly in medical settings where patients may have open wounds or lowered immune systems. While there are many available products that can kill such organisms efficiently, many of these products are harmful chemicals which can be toxic to humans if ingested and/or irritating/harmful to the touch, which is undesirable.

Additionally, many of the above noted bacteria can be found on agricultural products such as plants, herbs, vegetables, fruits, cannabis, hemp, etc. For instance E. coli has been frequently discovered in harmful quantities on lettuce plants. Additionally, powdery mildew is a fungus which can negatively affect various agricultural products. Conventional antimicrobial products cannot be applied to these types of products because they can be harmful to the agricultural product itself. Thus while conventional cleaning compositions may be effective at killing the bacteria and fungi on the agricultural product, conventional compositions can also potentially kill the underlying agricultural product. Agricultural products are also meant for human consumption, so toxic chemicals cannot safely be applied to such products.

Antiviral compositions also are needed. Compositions that can effectively kill viruses are also needed. Viruses, such as influenza, are endemic to the human population and causes illness and death worldwide every year. Additionally, new virus outbreaks present an ongoing threat to human and animal health, such as the novel COVID-19 virus, SARS, and MERS.

Accordingly, compositions that are effective against a variety of microbes are still needed.

### SUMMARY OF THE INVENTION

This Brief Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

One aspect of the disclosure is an antimicrobial composition comprising a water solution, where the water solution comprises a chlorite salt having a concentration ranging from about 2,000 parts per million to about 8,000 parts per million, and one or more (quaternary ammonium compounds (also referred to herein as “quits”) having a concentration ranging from about 5,000 parts per million to about 10,000 parts per million. In some embodiments, the composition can further comprise sodium tetraborate decahydrate (Borax) having a concentration of at least 8,000 parts per million, for example, about 8000 parts per million to about 15,000 parts per million. In some embodiments, the formula can include a surfactant having a concentration of at least about 1,000 parts per million. The Borax can also act a buffer for the antimicrobial composition and can also provide anti-fungal properties to the composition. The surfactant can decrease the surface tension of the anti-microbial composition to make it easier to apply to an object.

In one embodiment, an antimicrobial composition comprises a water solution comprising a chlorite salt having a concentration ranging from about 2,000 parts per million to about 8,000 parts per million, and at least one quaternary ammonium compound having a concentration ranging from about 5,000 parts per million to about 10,000 parts per million.

In some embodiments, the concentration of the chlorite salt to the water solution ranges from about 5,000 parts per million to about 8,000 parts per million, and the at least one quaternary ammonium salt has a concentration ranging from about 6,000 parts per million to about 10,000 parts per million.

In some embodiments, the quaternary ammonium salt comprises an n-alkyl dimethyl benzyl ammonium chloride, an n-alkyl dimethyl ethylbenzyl ammonium chloride, didecyldimethylammonium chloride, cetalkonium chloride, cetylpyridinium chloride, cetrimonium, tetraethylammonium bromide, domiphen bromide, benzethonium chloride, or any combination thereof.

In some embodiments, the quaternary ammonium salt comprises an n-alkyl dimethyl benzyl ammonium chloride and an n-alkyl dimethyl ethyl benzyl ammonium chloride. In some embodiments, the alkyl group on the n-alkyl dimethyl benzyl ammonium chloride comprises C₁₂, C₁₄, C₁₆ and Cis carbon groups. In some embodiments, the alkyl group on the n-alkyl dimethyl ethylbenzyl ammonium chloride comprises C₁₂ and C₁₄ carbon groups. In some embodiments, the n-alkyl dimethyl benzyl ammonium chloride comprises about 5% C₁₂, about 60% C₁₄, about 30% C₁₆, and about 5% C₁₈ carbon groups, and the n-alkyl dimethyl ethylbenzyl ammonium chloride comprises about 68% C₁₂ and about 32% C₁₄ carbon groups.

In some embodiments, the composition further comprises sodium tetraborate in a concentration ranging from about 8,000 parts per million to about 15,000 parts per million.

In some embodiments, the composition further comprises a buffer. In some embodiments, the buffer comprises sodium bicarbonate, ferric chloride, citric acid, sodium percarbonate, trisodium phosphate, acetic acid, sodium acetate, or any combination thereof.

In some embodiments, the buffer comprises the sodium acetate in a concentration ranging from about 500 to about 1500 parts per million. In some embodiments, the buffer further comprises acetic acid in a concentration ranging from about 100 to about 5000 parts per million, where the acetic acid has a dilution ratio of about 1:8 to about 1:12,

In some embodiments, the composition further comprises a surfactant having a concentration ranging from about 100 parts per million to about 3,000 parts per million. In some embodiments, the surfactant comprises non-ionic surfactant.

In some embodiments, the surfactant comprises a an alkoxylated non-ionic surfactant, such as ethoxylated alcohol. In some embodiments, the ethoxylated alcohols are C9-C11 ethoxylated alcohols.

In some embodiments, the pH of the composition ranges from about 6.8 to about 7.2.

In some embodiments, the antimicrobial composition has a sporicidal efficacy of substantially 100 percent against endospores of Clostridium difficile ATCC 43598 in an ASTM E2315 compliant test after a contact time up to about 120 seconds

In some embodiments, the antimicrobial composition has a sporicidal efficacy of substantially 100 percent against endospores of Escherichia Coli in an ASTM E2315 compliant test after a contact time of about 30 seconds.

In some embodiments, an antimicrobial comprises a water solution comprising: about 5,000 parts per million of a chlorite salt about 7,000 parts per million of a quaternary ammonium compound, and about 100 parts per million of an ethoxylated alcohol surfactant. In one embodiment, the quaternary ammonium compound comprises an n-alkyl dimethyl benzyl ammonium chloride and an n-alkyl dimethyl ethyl benzyl ammonium chloride. The composition may further comprises about 10,000 parts per million of sodium tetraborate In another embodiment, the composition further comprises about 800 parts per million of sodium acetate and about 3200 parts per million of acetic acid, wherein the acetic acid has a 1:10 dilution. In yet another embodiments, the composition further comprises sodium acetate, ferric chloride, citric acid, sodium percarbonate, trisodium phosphate, or any combination thereof in a concentration ranging from about 500 to about 1000 parts per million.

The anti-microbial composition can be used as a cleaning compound for killing undesirable bacteria on a desired surface or product, and can be sold in liquid or aerosol form in different embodiments. Thus, the present disclosure further provides methods for disinfecting an object comprising applying any of the afore described compositions to the object. The object may be a hard surface or a soft surface. In some embodiments, the object is contaminated with a bacteria or a virus, and the method kills at least 99% of the virus or bacteria on the object.

In another embodiment, the present disclosure provides a method for disinfecting air comprising electrostatically spraying any of the aforementioned compositions.

Another aspect of the present disclosure is applying an antimicrobial composition to an agricultural product, including but limited to plants, vegetables, fruits, herbs, grains, legumes, cannabis, hemp, etc. The antimicrobial composition can effectively eliminate the bacteria on the agricultural product while substantially preserving the agricultural product itself.

Another aspect of the present disclosure is applying an antimicrobial composition to a water supply to kill bacteria within the water supply such a method can be used in medical situations to clean treatment water, for instance in dialysis machines. The anti-microbial composition can effectively kill bacteria within the water but still be safe and consumable by a patient.

Numerous other objects, advantages and features of the present disclosure will be readily apparent to those of skill in the art upon a review of the following drawings and description of a preferred embodiment.

### BRIEF DESCRIPTION OF THE DRAWINGS

FIG. 1 depicts a table summarizing the kill results of an exemplary composition against P. aeruginosa.

FIG. 2 depicts a table summarizing the results of a study of an exemplary composition against E. coli and S. aureus.

FIG. 3 depicts a table summarizing the results of a study of an exemplary composition against E. aerogenes and S. aureus.

FIG. 4 depicts a table summarizing the results of a study of an exemplary composition against Acinetobacter baumannii.

FIG. 5 depicts a table summarizing the results of a study of an exemplary composition against Bordatella pertussis.

FIG. 6 depicts a table summarizing the results of a study of an exemplary composition against carbapenem resistant E. coli.

FIG. 7 depicts a table summarizing the results of a study of an exemplary composition against E. coli.

FIG. 8 depicts a table summarizing the results of a study of an exemplary composition against Klebsiella pneumoniae.

FIG. 9 depicts a table summarizing the results of a study of an exemplary composition against Legionella pneumophila.

FIG. 10 depicts a table summarizing the results of a study of an exemplary composition against Listeria monocytogenes.

FIG. 11 depicts a table summarizing the results of a study of an exemplary composition against methicillin-resistant S. aureus.

FIG. 12 depicts a table summarizing the results of a study of an exemplary composition against P. aeruginosa, Salmonella enterica, and S. aureus.

FIG. 13 depicts a table summarizing the results of a study of an exemplary composition against Trichophyton interdigitale.

FIG. 14 depicts a table summarizing the results of a study of an exemplary composition against vancomucin-resistant Enterococcus faecalis.

FIG. 15 depicts a table summarizing the results of a study of an exemplary composition against SARS-Related Coronavirus 2.

FIG. 16 depicts a table summarizing the results of a study of an exemplary composition against human coronavirus.

FIG. 17 is a schematic depicting an exemplary packing for dry ingredients for the present antimicrobial compositions.

### SUMMARY OF THE INVENTION

While the making and using of various embodiments of the present invention are discussed in detail below, it should be appreciated that the present invention provides many applicable inventive concepts that are embodied in a wide variety of specific contexts. The specific embodiments discussed herein are merely illustrative of specific ways to make and use the invention and do not delimit the scope of the invention. Those of ordinary skill in the art will recognize numerous equivalents to the specific apparatus and methods described herein. Such equivalents are considered to be within the scope of this invention and are covered by the claims.

In the drawings, not all reference numbers are included in each drawing, for the sake of clarity. In addition, positional terms such as “upper,” “lower,” “side,” “top,” “bottom,” etc. refer to the apparatus when in the orientation shown in the drawing. A person of skill in the art will recognize that the apparatus can assume different orientations when in use.

The qualifier “about” as used herein regarding contact time can describe a tolerance of up to five seconds around the stated contact time. The qualifier “about,” when used regarding sodium chlorite concentration describes a tolerance of up to five percent around the stated concentration.

The qualifier “substantially,” when used regarding percent of sporicidal efficacy, describes at least 99.99% of bacteria present in a sample being killed or eliminated, or any deviation from a 100 percent kill efficacy having no infectious impact and remaining within any associated applicable regulatory compliance.

The concentrations described herein are concentrations of a particular component with respect to the complete antimicrobial composition, not only the water component of the composition.

One aspect of the present disclosure is an antimicrobial composition including a chlorite salt dissolved in water. The present compositions include a chlorite salt, such as sodium chlorite, as an ingredient. Those skilled in the art will readily appreciate that some of the sodium chlorite may, upon dissolution in water, form chlorine dioxide.

In some embodiments, the anti-microbial composition can have a demonstrable sporicidal efficacy of substantially 100 percent against endospores of Clostridium difficile ATCC 43598 in an ASTM E2315 compliant test after a contact time up to about 120 seconds, or after about 120 seconds.

In some embodiments, the concentration of sodium chlorite to water solution can be between about 1,000 parts-per-million and about 10,000 parts per million. In some embodiments, the concentration of sodium chlorite to water solution can be between about 3,000 parts-per-million and about 7,000 parts per million. In some embodiments, the concentration of sodium chlorite to water solution can be between about 4,000 parts-per-million and about 6,000 parts per million. In some embodiments, the concentration of sodium chlorite to water solution can be about 1,000 parts-per-million, 2,000 parts-per-million, 3,000 parts-per-million, 4,000 parts-per-million, 5,000 parts-per-million, 6,000 parts-per-million, 7,000 parts-per-million, 8,000 parts-per-million, 9,000 parts-per-million, or 10,000 parts-per-million.

The sodium chlorite sodium chlorite in the anti-microbial composition can provide anti-microbial or anti-bacterial properties to effectively bacteria, viruses, mold, and other microbial pathogens in contact with the anti-microbial composition, such as Clostridium difficile, Staphylococcus aureus, Escherichia coli, Pseudomonas aeruginosa, Enterobacter aerogenes, Acinetobacter baumanni, B. pertussis, E. coli, carbapenem-resistant E. coli, K. pneumonia, Legionella. pneumophila, Listeria monocytogenes, methicillin resistant S. aureus, P. aeruginosa, Salmonella enterica, S. aureus, Trichophyton interdigitale, vancomycin-resistant Enterococcus faeclis, and others.

The quarternary ammonium compound included in the formulation also contributes anti-microbial activity. While not being bound by theory, it is believed that the quarternary ammonium compound and the sodium chlorite act synergistically to enhance the anti-microbial effects.

In some embodiments, the contact time to produce a sporicidal efficacy of substantially 100 percent against endospores of Clostridium difficile ATCC 43598 in an ASTM E2315 compliant test can range from about 60 seconds to about 120 seconds. In some embodiments, the contact time can range from about 30 seconds to about 120 seconds. In some embodiments, the contact time to produce a sporicidal efficacy of substantially 100 percent against endospores of Clostridium difficile ATCC 43598 in an ASTM E2315 compliant test can be about 15 seconds. In some embodiments, the antimicrobial composition can have a demonstrable sporicidal efficacy of substantially 100 percent against endospores of other bacteria such as Staphylococcus aureus, Escherichia coli, Pseudomonas Aeruginosa, etc. in an ASTM E2315 compliant test after a contact time of about 15-120 seconds.

In some embodiments, the antimicrobial composition can include sodium chlorite and a quaternary ammonium compound solution. In some embodiments the antimicrobial composition can have a demonstrable sporicidal efficacy of substantially 100 percent against endospores of Clostridium difficile ATCC 43598 in an ASTM E2315 compliant test after the contact times mentioned herein. The one or more quats may provide additional antimicrobial or anti-bacterial properties, which can help kill unwanted or harmful bacteria.

In some embodiments, the total concentration of the one or more Quats to the water solution can range between about 4,000 parts per million and about 12,000 parts per million. In some embodiments, the total concentration of the one or more quats to the water solution can range between about 6,000 parts per million and about 10,000 parts per million. In some embodiments, the total concentration of the one or more quats to the water solution can range between about 6,000 parts per million and about 9,000 parts per million. In some embodiments, the total concentration of the one or more quats to the water solution can be about 4,000 parts per million, 5,000 parts per million, 6,000 parts per million, 7,000 parts per million, 8,000 parts per million, 9,000 parts per million, or 10,000 parts per million, 11,000 parts per million, or 12,000 parts per million. In some embodiments a single Quat can be used, while in other embodiments multiple quats can be used in combination with one another.

Quats can provide biocidal properties against bacteria by disrupting cell walls of the bacteria. Various types of Quats can be used, including but not limited to the quaternary ammonium salt comprises an n-alkyl dimethyl benzyl ammonium chloride, an n-alkyl dimethyl ethylbenzyl ammonium chloride, didecyldimethylammonium chloride, cetalkonium chloride, cetylpyridinium chloride, cetrimonium, tetraethylammonium bromide, domiphen bromide, benzethonium chloride, or any combination thereof.

In some embodiments, in some embodiments, the quaternary ammonium salt comprises an n-alkyl dimethylbenzylammonnium chloride and an n-alkyl dimethylethylbenzyl ammonium chloride. The n-alkyl group of the n-alkyl dimethylbenzylammonnium chlorides may comprise n-alkyl groups chosen from C12, C14, C16, C18, and any combination thereof. The n-alkyl group of the n-alkyl dimethylethylbenzylammonnium chlorides may comprise n-alkyl groups chosen from C12, C14, and combinations thereof. The antimicrobial composition of claim 4, wherein the n-alkyl dimethyl benzyl ammonium chloride comprises about 5% C12, about 60% C14, about 30% C16, and about 5% C18 carbon groups, and the n-alkyl dimethyl ethylbenzyl ammonium chloride comprises about 68% C12 and about 32% C14 carbon groups.

In some embodiments, the quaternary ammonium compounds are BTC® 2125M, available from Stephan Antimicrobials.

In some embodiments, the anti-microbial composition can further include sodium tetraborate decahydrate (Borax). Borax can act as a buffer to help balance the pH of the antimicrobial composition. Borax may also provide anti-fungal characteristics to help kill and prevent fungi from growing on a surface to be cleaned. In some embodiments, the concentration of the Borax in the water solution can range from about 5,000 parts per million and about 15,000 parts per million, or about 8,000 parts per million to about 15,000 parts per million. In some embodiments, the concentration of sodium tetraborate in the composition ranges from about 7,000 parts per million to about 13,000 parts per million. In some embodiments, the concentration of sodium tetraborate in the composition ranges from about 9,000 parts per million to about 11,000 parts per million. In some embodiments, the concentration of sodium tetraborate in the composition is about 5,000 parts-per-million, about 6,000 parts per million, about 7,000 parts per million, about 8,000 parts per million, about 9,000 parts per million, about 10,000 parts per million, about 11,000 parts-per-million, about 12,000 parts-per-million, about 13,000 parts per million, about 14,000 parts-per-million, or about 15,000 parts per million.

In further embodiments, the antimicrobial composition further comprises a buffer chosen from sodium bicarbonate, ferric chloride, citric acid, sodium percarbonate, trisodium phosphate, acetic acid, sodium acetate, and any combination thereof. The buffer may be in addition to the sodium tetraborate, or the composition may comprise one of these buffers, and not include sodium tetraborate.

In some embodiments, the composition does not comprise sodium tetraborate, and further comprises a buffer chosen from sodium bicarbonate, ferric chloride, citric acid, sodium percarbonate, trisodium phosphate, and any combination thereof.

In some embodiments, the antimicrobial composition can include sodium acetate and acetic acid as a buffer. The acetic acid may have a dilution ratio 1:5 and 1:15. In some embodiments, the concentration of the sodium acetate in the water solution can range from about 500 parts per million to about 1500 parts per million. In some embodiments, the concentration of the sodium acetate to the water solution can range from about 600 parts per million to about 1100 parts per million. In some embodiments, the concentration of the sodium acetate in the water solution can range from about 700 parts per million to about 900 parts per million. In some embodiments, the concentration of the sodium acetate in the water solution can be about 500 parts-per-million, about 600 parts per million, about 700 parts per million, about 800 parts per million, about 900 parts per million, or about 1000 parts per million, about 1100 parts-per-million, about 1200 parts-per-million, about 1300 parts per million, about 1400 parts-per-million, or about 1500 parts per million.

In some embodiments, the dilution ratio of the acetic acid can range between about 1:8 and 1:12. In some embodiments, the dilution of acetic acid can be about 1:5, 1:6, 1:7, 1:8, 1:9, 1:10, 1:11, 1:12, 1:13, 1:14, or 1:15.

In some embodiments, the antimicrobial composition can include a chlorite salt, a quat, and a non-reactive surfactant. In some embodiments, the nonreactive surfactant is a non-amine surfactant. In other embodiments, the nonreactive surfactant can be a non-octyldimethylamine oxide surfactant. In other embodiments nonreactive surfactant is a non-lauryldimethylamine oxide surfactant. In still other embodiments the surfactant can be any suitable surfactant which can help decrease the surface tension of the antimicrobial composition to allow for easier dispersion of the antimicrobial compound on a surface or product of interest, In some embodiments, the surfactant can be an alkoxylated non-ionic surfactant, such as ethoxylated alcohols. In some embodiments, the ethoxylated alcohols comprise C₆-C₂₀ ethoxylated alcohols, while in other embodiments, the ethoxylated alcohols comprise C₉-C₁₁ ethoxylated alcohols, including those surfactants sold under the brand name Tomadol®, such as Tomadol 900. In other embodiments, the surfactant can include nonyl phenol ethoxylates, nonyl phenol propoxylates, or linear alkoxylated C6-C20 alcohols (4 mol-15 mol EO or PO).

In some embodiments, the concentration of the surfactant in the water solution can range from about 100 parts per million to about 3000 parts per million. In some embodiments, the concentration of the surfactant in the water solution can range from about 500 parts per million and about 2000 parts per million. In some embodiments, the concentration of the surfactant in the water solution can range from about 700 parts per million to about 1300 parts per million. In some embodiments, the concentration of the surfactant in the water solution can be about 300 parts-per-million, about 400 parts per million, about 500 parts per million, about 600 parts per million, about 700 parts-per-million, about 800 parts per million, about 900 parts per million, about 1000 parts per million, about 1100 parts per million, about 1100 parts per million, about 1200 parts-per-million, about 1300 parts-per-million, about 1400 parts per million, about 1500 parts-per-million, about 1600 parts per million, about 1700 parts per million, about 1800 parts per million, about 1900 parts-per-million, or about 2000 parts per million.

In some embodiments, the antimicrobial composition can include certain components having the following weight percentages: 98.4%-99.0% water, 0.45%-0.55% sodium chlorite, and 0.63%-0.77% quats. In some embodiments, the antimicrobial composition can further include additional components having the following weight percentages: 0.009%-0.011% acetic acid, 0.09%-0.11% surfactant, and 0.072%-0.088% sodium acetate. In some embodiments, the sodium tetraborate can be substituted by any one of the following components in the amount of 0.072%-0.088% by weight: baking soda (sodium bicarbonate, NaHCO₃), iron chloride (FeCl₃), citric acid (C₆H₈O₇), sodium percarbonate(Na₂H₃CO₆), trisodium phosphate (Na₃PO₄).

In some embodiments, the antimicrobial composition of the present disclosure can be sold in a powder form that can be subsequently added to an appropriate amount of water. In some embodiments, the powder antimicrobial composition can have the certain components in the following dry weight percentages: 30.0%-47.0% sodium chlorite, and 45.0%-63.0% quats. In some embodiments, the powder antimicrobial composition can have the certain components in the following weight percentages: 30.0%-40.0% sodium chlorite, 45.0%-55.0% quats, 0.5%-0.9% acetic acid, 5.0%-0.9.0% surfactant, and 4.0%-0.7.0% sodium acetate (or the other substitutes identified above.

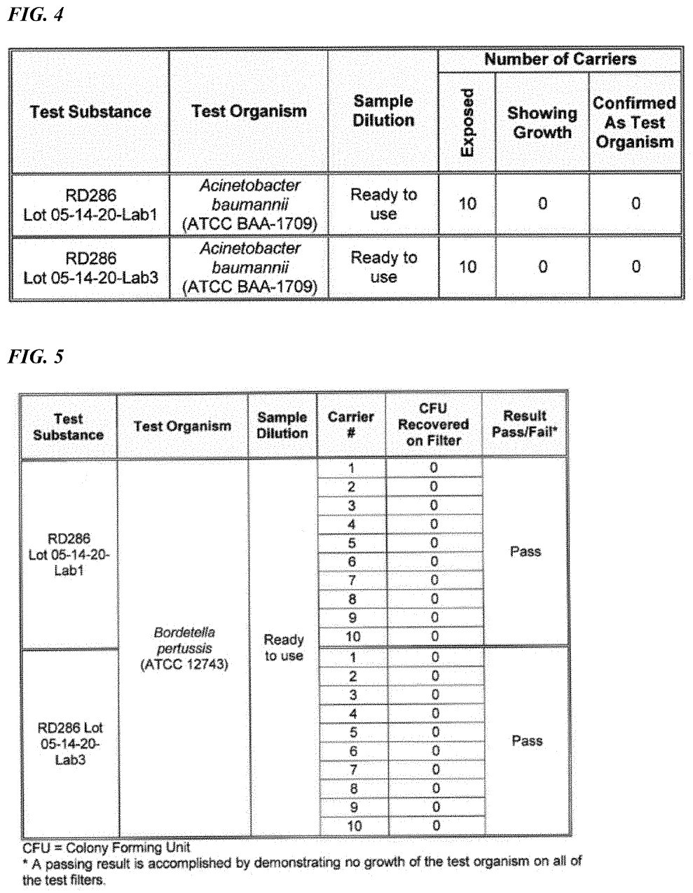

In some embodiments, as shown in FIGS. 4-5, the powder can be packaged into multiple compartment containers with some components of the powder separated from others until the powder is added to water. For instance, in some embodiments, the sodium chlorite can be kept separate from the other components of the powder within the packaging for the powder. The packaging can be torn and the contents poured into an appropriate amount of water to form the desired antimicrobial solution. Having separate compartments for one or more components of the powder antimicrobial composition can help prevent unwanted chemical reactions between the powder chemical components prior to mixing the powder antimicrobial composition with water, thereby prolonging shelf life and advantageously providing a more portable product. An appropriate amount of water in some embodiments can be defined as an amount of water that when combined with the power antimicrobial composition produces a solution 98.4%-98.8% water by weight and 1.2-1.6% powder components by weight.

In another embodiment, the antimicrobial composition can be formulated as a hand care product, such as a disinfecting gel, spray, wipe, or lotion. In one embodiment, the composition comprises sodium chlorite in an amount ranging from about 0.4-0.5% by weight, quates (such as Stepan BTC® 2125 (80%)) in an amount ranging from about 0.6 to about 0.7% by weight, a surfactant (such as Tomadol® 900) in an amount ranging from about 0.05% to about 0.1% by weight, sodium tetraborate in an amount ranging from about 0.5% to about 1.0% by weight, an emollient compound in an amount ranging from about 0.1 to about 0.5% by weight, and up to about 97.5% by weight of deionized water. The emollient compound may be, in one embodiment, glycerin. In other embodiments, the emollient comprise glycerin shea butter, cocoa butter, lanolin, or any combination thereof. In some embodiments, the sodium tetraborate may be substituted by any one of the following components in the amount of 0.072%-0.088% by weight: baking soda (sodium bicarbonate, NaHCO₃), iron chloride (FeCl₃), citric acid (C₆H₈O₇), sodium percarbonate(Na₂H₃CO₆), trisodium phosphate (Na₃PO₄). The hand care formulation may be advantageously packaged for various uses, such as in dispensers for health care setting, public restroom settings, personal dispensing bottles for travel, etc.

The antimicrobial composition can be used in many different types of applications or administration protocols. For instance, in some embodiments, the antimicrobial composition is a hard surface disinfectant, and can come in a liquid form, which can be poured or sprayed from a conventional spray bottle onto a desired surface to be cleaned. In other embodiments, the antimicrobial composition can come in aerosol form and be an air disinfectant. In some embodiments, the antimicrobial composition can be an electrostatically sprayable air disinfectant. The anti-microbial composition can be used to clean surfaces in a variety of environments, including but not limited to medical, industrial, and residential environments (kitchens, bathrooms, etc.), hospitals, medical facilities, medical clinics, schools or other public buildings, industrial packaging plants, factories, manufacturing facilities, food processing and packaging facilities, restaurants, bars, etc. The antimicrobial composition can also be utilized as a wound cleaner or an antiseptic for cleaning out cuts, abrasions, or other wounds as well as to sterilize an injection or surgical site in a healthcare setting. The antimicrobial composition can also be used for industrial cleaning services, such as for mold and mildew removal services.

The above-described compositions may be used in the concentrations described above, or, if desired and depending on the application, the composition may be further diluted. For example, the composition may be diluted by an end user with additional water in an amount ranging from about 1:1 to about 1:40, about 1:1 to about 1:20, about 1:2 to about 1:20, about 1:2 to about 1:15, about 1:5 to about 1:20, about 1:5 to about 1:15, about 1:10, about 1:25, about 1:20, or about 1:40. Alternatively, the antimicrobial composition may be diluted and sold in ready-to-use forms for particular indications.

Another aspect of the present invention is a method of treating an agricultural product comprising the steps of: providing an antimicrobial composition, including any of the above described compositions, and applying the anti-microbial composition on the agricultural product. The anti-microbial can effectively kill undesirable bacteria from the agricultural product while leaving the agricultural product substantially intact. In some embodiments, the antimicrobial composition is diluted prior to treatment at a dilution ratio of about 1:10 to about 1:40. In some embodiments, the agricultural product can be plants of various varieties, vegetables, fruits, legumes, grains, cannabis, hemp etc. The antimicrobial composition of the present disclosure can advantageously kill unwanted bacteria and/or fungi while preserving the integrity of the underlying agricultural product. Testing of one embodiment of the antimicrobial composition on cannabis plants showed that the antimicrobial composition provided the sporicidal efficacies discussed herein while no significant damage or negative effect was observed in the cannabis plants to which the compound was applied. In still other embodiments, the anti-microbial composition can be applied to the meat and poultry industry to clean meat and poultry products prior to packaging.

Another aspect of the present invention is a method of treating a food source, such as that to be fed to animals or livestock, comprising the steps of: providing any of the above-described antimicrobial compositions and applying the anti-microbial composition on the food source. Applying the antimicrobial composition to a food source such as animal feed products can help kill any unwanted bacteria in the food source prior to the food source being fed to the animal or livestock. Having the food source treated with the antimicrobial composition of the present disclosure has also been shown to kill harmful bacteria inside the belly or digestive track of the target animal once the food source is ingested, including in chickens and pigs. Such a treatment protocol can help keep the animals or livestock healthy and help prevent unwanted bacteria to be passed on to humans who may consume any such animals or livestock.

Another aspect of the present disclosure is a method of treating a water supply including the steps of: providing an antimicrobial composition as described above; and introducing the anti-microbial composition into the water supply. In some embodiments, the antimicrobial composition can meet the EPA standards for a Category IV product or be non-toxic and non-irritant from a regulatory standpoint. As such, the anti-microbial can be consumed safely by humans and animals such that the antimicrobial composition can be used to treat drinking water supplies or other water supplies which can interact with humans and animals. In a medical setting, the antimicrobial composition can be used to treat water supplies which can be provided to varying medical devices, e.g. a medical dialysis unit.

The antimicrobial composition of the present disclosure can thus provide antimicrobial properties which can help kill unwanted bacteria from a surface or product. In some embodiments and application, the antimicrobial composition can also help provide antibacterial, antifungal, sanitization, disinfectant, odor elimination, or other beneficial cleaning characteristics. The anti-microbial composition can also generally be safe for contact with humans and animals, such that the product can be used to treat agricultural products and or water supplies which may be safely consumed or utilized by the public.

### EXEMPLARY EMBODIMENTS

1. An antimicrobial composition comprising: a water solution comprising a chlorite salt having a concentration ranging from about 2,000 parts per million to about 8,000 parts per million, and at least one quaternary ammonium salt having a concentration ranging from about 5,000 parts per million to about 10,000 parts per million.
2. The antimicrobial composition of embodiment 1, wherein: the concentration of the chlorite salt to the water solution ranges from about 5,000 parts per million to about 8,000 parts per million, and the at least one quaternary ammonium salt has a concentration ranging from about 6,000 parts per million to about 10,000 parts per million.
3. The antimicrobial composition of embodiment 1 or 2, wherein the quaternary ammonium salt comprises an n-alkyl dimethyl benzyl ammonium chloride, an n-alkyl dimethyl ethylbenzyl ammonium chloride, didecyldimethylammonium chloride, cetalkonium chloride, cetylpyridinium chloride, cetrimonium, tetraethylammonium bromide, domiphen bromide, benzethonium chloride, or any combination thereof 4. The antimicrobial composition of embodiment 3, wherein the quaternary ammonium salt comprises an n-alkyl dimethyl benzyl ammonium chloride and an n-alkyl dimethyl ethyl benzyl ammonium chloride.
5. The antimicrobial composition of embodiment 4, wherein the alkyl group on the n-alkyl dimethyl benzyl ammonium chloride comprises C₁₂, C₁₄, C₁₆ and Cis carbon groups.
6. The antimicrobial composition of embodiment 4, wherein the alkyl group on the n-alkyl dimethyl ethylbenzyl ammonium chloride comprises C₁₂ and C₁₄ carbon groups.
7. The antimicrobial composition of embodiment 4, wherein the n-alkyl dimethyl benzyl ammonium chloride comprises about 5% C₁₂, about 60% C₁₄, about 30% C₁₆, and about 5% C₁₈ carbon groups, and the n-alkyl dimethyl ethylbenzyl ammonium chloride comprises about 68% C₁₂ and about 32% C₁₄ carbon groups
8. The antimicrobial composition of any one of embodiments 1 to 7, further comprising sodium tetraborate in a concentration ranging from about 8,000 parts per million to about 15,000 parts per million.
9. The antimicrobial composition of any one of embodiments 1 to 8, further comprising a buffer.
10. The antimicrobial composition of embodiment 9, wherein the buffer comprises sodium bicarbonate, ferric chloride, citric acid, sodium percarbonate, trisodium phosphate, acetic acid, sodium acetate, or any combination thereof.
11. The antimicrobial composition of embodiment 10, wherein the buffer comprises the sodium acetate in a concentration ranging from about 500 to about 1500 parts per million.
12. The antimicrobial composition of embodiment 10 or 11, wherein the buffer further comprises acetic acid in a concentration ranging from about 100 to about 5000 parts per million, where the acetic acid has a dilution ratio of about 1:8 to about 1:12.
13. The antimicrobial composition of any one of embodiments 1 to 12, 1, further comprising a surfactant having a concentration ranging from about 100 parts per million to about 3,000 parts per million.
14. The antimicrobial composition of embodiment 13, wherein the surfactant comprises a non-ionic surfactant.
15. The antimicrobial composition of embodiment 13, wherein the surfactant comprises an alkoxylated non-ionic surfactant.
16. The antimicrobial composition of embodiment 15, wherein the alkoxylated non-ionic surfactant comprises ethoxylated alcohols.
17. The antimicrobial composition of embodiment 16, wherein the ethoxylated alcohols are C₉-C₁₁ ethoxylated alcohols.
18. The antimicrobial composition of any one of embodiments 1 to 17, wherein the pH of the composition ranges from about 6.8 to about 7.2.
19. The antimicrobial composition of any one of embodiments 1 to 18, wherein the antimicrobial composition has a sporicidal efficacy of substantially 100 percent against endospores of Clostridium difficile ATCC 43598 in an ASTM E2315 compliant test after a contact time up to about 120 seconds.
20. The antimicrobial composition of any one of embodiments 1 to 19, wherein the antimicrobial composition has a sporicidal efficacy of substantially 100 percent against endospores of Escherichia Coli in an ASTM E2315 compliant test after a contact time of about 30 seconds.
21. An antimicrobial composition comprising water solution comprising:

about 5,000 parts per million of a chlorite salt

about 7,000 parts per million of a quaternary ammonium compound, and

about 100 parts per million of an ethoxylated alcohol surfactant.

22. The antimicrobial composition of embodiment 21, wherein the quaternary ammonium compound comprises an n-alkyl dimethyl benzyl ammonium chloride and an n-alkyl dimethyl ethyl benzyl ammonium chloride.
23. The antimicrobial composition of embodiment 21 or 22, further comprising about 10,000 parts per million of sodium tetraborate.
24. The antimicrobial composition of any one of embodiments 21 to 23, further comprising about 800 parts per million of sodium acetate and about 3200 parts per million of acetic acid, wherein the acetic acid has a 1:10 dilution.
25. The antimicrobial composition of any one of embodiments 21 to 24, wherein the composition further comprises sodium acetate, ferric chloride, citric acid, sodium percarbonate, trisodium phosphate, or any combination thereof in a concentration ranging from about 500 to about 1500 parts per million.
26. A method for disinfecting an object comprising applying the composition of claim 1 to the object.
27. The embodiment of claim 26, wherein the object is a hard surface or a soft surface.
28. The embodiment of claim 28, wherein the object is contaminated with a bacteria or a virus, and the method kills at least 99.5% of the virus or bacteria on the object.
29. The embodiment of claim 28, wherein the bacteria comprises Clostridium difficile, Staphylococcus aureus, Escherichia coli, Pseudomonas Aeruginosa, Enterobacter aerogenes, or any combination thereof.
30. The embodiment of claim 28, wherein the virus comprises COVID-19, SARS, MERS, influenza, or any combination thereof.
31. A method of disinfecting an agricultural product comprising applying the composition of any one of embodiments 1 to 25 to the agricultural product.
32. The method of embodiment 31, wherein the method substantially kills fungus, mold, spores, bacteria, or viruses on the agricultural product.
33. The method of embodiment 31 or 32, wherein the agricultural product in a cannabis plant.
34. An antimicrobial hand care composition comprising sodium chlorite in an amount ranging from about 0.4-0.5% by weight, a quaternary ammonium salt in an amount ranging from about 0.6 to about 0.7% by weight, a surfactant in an amount ranging from about 0.05% to about 0.1% by weight, sodium tetraborate in an amount ranging from about 0.5% to about 1.0% by weight, an emollient compound in an amount ranging from about 0.1 to about 0.5% by weight, and up to about 97.5% by weight of deionized water.
35. The antimicrobial hand care composition of embodiment 34 comprising sodium chlorite in an amount ranging from about 0.4-0.5% by weight, a quaternary ammonium salt in an amount ranging from about 0.6 to about 0.7% by weight, a surfactant in an amount ranging from about 0.05% to about 0.1% by weight, baking soda (sodium bicarbonate, NaHCO₃), iron chloride (FeCl₃), citric acid (C₆H₈O₇), sodium percarbonate(Na₂H₃CO₆), trisodium phosphate (Na₃PO₄) in an amount ranging from about 0.07% to about 0.88% by weight %, an emollient compound in an amount ranging from about 0.1 to about 0.5% by weight, and up to about 97.5% by weight of deionized water.
36. The antimicrobial hand care composition of embodiment 34 or 35, wherein the emollient compound comprises glycerin, shea butter, cocoa butter, lanolin, propylene glycol, or any combination thereof.
37. The antimicrobial hand care composition of embodiments 34 to 36, wherein the quaternary ammonium compound comprises the quaternary ammonium salt comprises an n-alkyl dimethyl benzyl ammonium chloride, an n-alkyl dimethyl ethylbenzyl ammonium chloride, didecyldimethylammonium chloride, didecyldimethylammonium bromide, cetalkonium chloride, cetalkonium bromide, cetylpyridinium chloride, cetylpyridinium bromide, cetyltrimethylammonium bromide, cetyltrimethylammonium chloride, cetrimonium, tetraethylammonium bromide, domiphen bromide, domiphen chloride, dofanium chloride, benzethonium chloride, benzyl(C₁₂₋₁₈)alkyldimethylammonium chloride, benzyldodecyldimethylammonium bromide, benzyldodecyldimethylammonium chloride, dodecyltrimethylammonium bromide, dodecyltrimethylammonium chloride, hexadecyltrimethylammonium bromide, hexadecyltrimethylammonium chloride, methylbenzethonium chloride, tetradecyltrimethylammonium bromide, tetradecyltrimethylammonium chloride, tetraethylammonium bromide, tetraethylammonium chloride, or any combination thereof.

### EXAMPLES

### Example 1

Table 1 lists exemplary antimicrobial compositions in accordance with the present disclosure.

TABLE 1
Exemplary Formulations (ingredients are listed as
weight percent, with ranges listed in parentheses).

| Ingredient | Formula 1 | Formula 2 | Formula 3 | |
|---|---|---|---|---|
| Water | 97.7% | 97.30 | | |
| Sodium chlorite | 0.5% (0.45-0.55) | 0.5% (0.45-0.55) | 0.5% | (0.45-0.55) |
| Stepan BTC ® | 0.7% (0.63-0.77) | 0.7% (0.63-0.77) | 0.7% | (0.63-0.77) |
| 2125-80% | | | | |
| Tomadol ® 900 | 0.1% (0.09-0.11) | 0.1% (0.09-0.11) | 0.1% | (0.09-0.11) |
| Sodium Tetraborate | 1.0% (0.90-1.10) | 1.0% (0.90-1.10) | | |
| decahydrate | | | | |
| Sodium acetate | | 0.08% (0.07-0.09) | 0.08% | (0.07-0.09) |
| Glacial acetic acid | | 0.32 (0.32-0.33) | 0.01% | (0.009-0.011) |
| (1:10 dilution) | | | | |

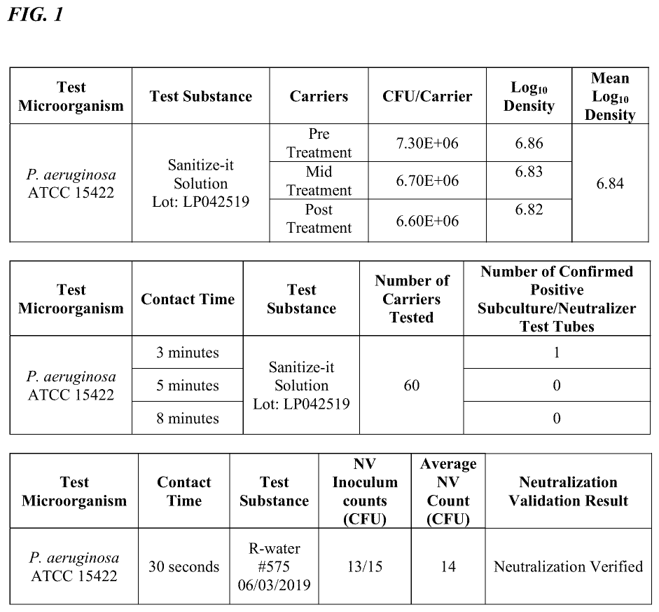

FIG. 1 depicts a table summarizing results from kill tests of a composition of the present disclosure comprising 0.5% sodium chlorite when tested against P. aeruginosa. Additional testing has confirmed that the Formula 1 can kill E. coli, S. aureus, and Botrytis cinerea at a sporicidal efficacy of 99.9999% after 15 seconds, and can kill P. aeruginosa at a sporicidal efficacy of 99.99% after 15 seconds.

### Example 2: Activity Against S. aureus and E. coli

The purpose of this assay is to determine the efficacy of Formula 1 (RD286) to sanitize pre-cleaned, nonporous food contact surfaces using the AOAC Germicidal and Detergent Sanitizing Action of Disinfectants method. This method is in compliance with the requirements of the U.S. Environmental Protection Agency (EPA) and Health Canada.

Preparation of Test Substance: An equivalent dilution of 1:15, defined as 1 part test substance+15 parts diluent, was prepared using 14.0 ml of the test substance and 210.0 ml of 400 ppm AOAC Synthetic Hard Water. The prepared test substance was homogenous as determined by visual observation and was used within three hours of preparation. A 99.0 ml aliquot of test substance was transferred to a sterile 250-300 ml Erlenmeyer flask per test organism, per lot. Each flask was placed into a water bath at 25.0° C. and equilibrated for ≥10 minutes.

Preparation of Test Organisms: For Staphylococcus aureus (ATCC 6538) and Escherichia coli (ATCC 11229), a loopful of a thawed cryovial of stock organism broth culture was streaked to a Nutrient Agar A slant medium and was incubated at 35-37° C. (36.0° C.) for 24±2 hours (23 hours). For the final test culture, 5.0 ml of Phosphate Buffer Dilution Water (PBDW) was added to the Nutrient Agar A slant, following incubation. Using a sterile loop, the growth was dislodged from the agar surface. The mixture was collected, transferred to a vessel containing 99.0 ml of PBDW and mixed thoroughly. A total of 5 Nutrient Agar B plates were inoculated, per test organism, using 200 μl of culture, spreading the inoculum to create a lawn of growth. The plates were incubated at 35-37° C. (36.0° C.) for 24±2 hours (24 hours). Following incubation, 5.0 ml of Phosphate Buffered Saline+0.1% Tween 80 was added to each plate. Using a plate spreader, the culture was gently dislodged from the agar surface avoiding disrupting the agar. The culture was collected, combined, and then mixed thoroughly. The collected culture was filtered through sterile Whatman #2 filter paper using a vacuum source. To target approximately 1×109 to 1×10 1° C.FU/ml (9-10 logs/ml}, a spectrophotometric analysis was performed using a wavelength of 620 nm. The final absorbance value was 1.443 for Staphylococcus aureus (ATCC 6538) and 1.441 for Escherichia coli (ATCC 11229).

Addition of Organic Soil Load: A 0.30 ml aliquot of FBS was added to 5.7 ml of each prepared culture to yield a 5% fetal bovine serum organic soil load.

Exposure Conditions: Each flask containing the test substance was whirled stopping just before the suspension was added, creating enough residual motion of liquid to prevent pooling of the suspension at the point of contact with test substance. A 1.00 ml aliquot of culture was added midway between the center and edge of the surface with the tip of the pipette slightly immersed in the test solution. Touching the neck or side of the flasks was avoided. Each flask was swirled to thoroughly mix the contents and was exposed for the 30 seconds exposure time at the exposure temperature 25±1 (25.0° C.).

Test System Recovery: Following exposure, 1.00 ml of the inoculated test substance was transferred to 9 ml of neutralizer. The neutralized material was vortex mixed. The neutralized contents corresponded to the 10-1 dilution. Four 1.00 ml and four 0.100 ml aliquots of the neutralized material were transferred to individual sterile Petri dishes spread-plated onto the subculture agar medium.

Incubation and observation: All subculture plates were incubated for 24-30 hours (24 hours) at 35-37° C. (36.0° C.). Following incubation, the subculture plates were visually examined for growth. Representative test and positive control subcultures showing growth were visually examined, Gram stained and biochemically assayed to confirm or rule out the presence of the test organism.

Purity Control: A “streak plate for isolation” was performed on each organism culture and following incubation examined in order to confirm the presence of a pure culture. The acceptance criterion for this study control is a pure culture demonstrating colony morphology typical of the test organism.

Organic Soil Sterility Control: Concurrent with testing, the serum used for the organic soil load was cultured, incubated, and visually examined for growth. The acceptance criterion for this study control is lack of growth.

Neutralizer Sterility Control: Concurrent with testing, the neutralizer used in testing was evaluated for sterility. A representative sample of neutralizer (1.00 ml) was plated onto the subculture medium as in the test. The plate was incubated and visually examined. The acceptance criterion for this study control is a lack of growth.

Test Substance Diluent Sterility Control: Concurrent with testing, the test substance diluent used in testing was evaluated for sterility. A representative sample of test substance diluent (1.00 ml) was plated onto the subculture agar medium as in the test. The plate was incubated and visually examined. The acceptance criterion for this study control is a lack of growth.

PBDW Sterility Control: Concurrent with testing, the PBDW used in testing was evaluated for sterility. A representative sample of PBDW (1.00 ml) was plated onto the subculture medium as in the test. The plate was incubated and visually examined. The acceptance criterion for this study control is a lack of growth.

Test Substance Sterility Control: A representative sample of prepared test substance (1.00 ml), per lot used in testing, was plated onto the subculture agar medium as in the test. Each plate was incubated and visually examined.

Numbers Control: A 99.0 ml aliquot of PBDW was transferred to a sterile 250-300 ml Erlenmeyer flask, per test organism. Each flask was equilibrated in a water bath at 25.0° C. for ≥10 minutes. Each flask was whirled and 1.00 ml of culture was added as in the test procedure. Each flask was swirled to thoroughly mix the contents. Within approximately 30 seconds, 1.00 ml of the contents was transferred to 9 ml of neutralizer. The neutralized contents correspond to the 10-1 dilution. Ten-fold serial dilutions were prepared to 10·6. Four 1.00 ml and four 0.100 ml aliquots of the 10-e dilution were plated onto the subculture agar medium as in the test. This resulted in the 10⁻⁶ and 10⁻⁷ dilutions, respectively. The plates were incubated. The acceptance criterion for this control is a minimum value of 7.0 log₁₀.

Neutralization Confirmation Control: The following neutralization confirmation control was performed concurrent with testing. Each prepared test culture was diluted to target 1×10⁴-1×10⁵ CFU/ml (to target a result of 10-100 CFU plated in each control run). Multiple organism dilutions were prepared.

Test Culture Titer (TCT): A 0.100 ml aliquot of diluted test organism was added to 10.0 ml of PBDW and was vortex mixed. The mixture was held for a minimum of 2 minutes and duplicate 0.100 ml aliquots were spread plated as in the test. The acceptance criterion for this study control is growth.

Neutralization Confirmation Control Treatment (NCT): A 1.00 ml aliquot of test substance, per lot, was added to 9 ml of neutralizer and was vortex mixed. Within approximately 30 seconds, 0.100 ml of diluted test organism was added to the neutralized contents and was vortex mixed. The mixture was held for a minimum of 2 minutes and duplicate 0.100 ml aliquots were spread plated as in the test. The acceptance criterion for this study control is growth within 1 log₁₀ of the test culture titer (TCT).

Neutralizer Toxicity Treatment (NTT): A 0.100 ml aliquot of diluted test organism was added to 10.0 ml of neutralizer and was vortex mixed. The mixture was held for a minimum of 2 minutes and duplicate 0.100 ml aliquots were spread plated as in the test. The acceptance criterion for this study control is growth within 1 log₁₀ of the test culture titer (TCT).

Lots 1, 2 and 3 of Formula 1, diluted 1:15, defined as 1 part test substance+15 parts 400 ppm AOAC Synthetic Hard Water, demonstrated a 99.9999% (6.04 Log₁₀), >99.99999% (>7.52 Log₁₀) and >99.99999% (>7.52 Log₁₀) reduction of Escherichia coli (ATCC 11229), respectively, following a 30 second exposure time at 25±1 (25.0° C.) in the presence of a 5% fetal bovine serum organic soil load.

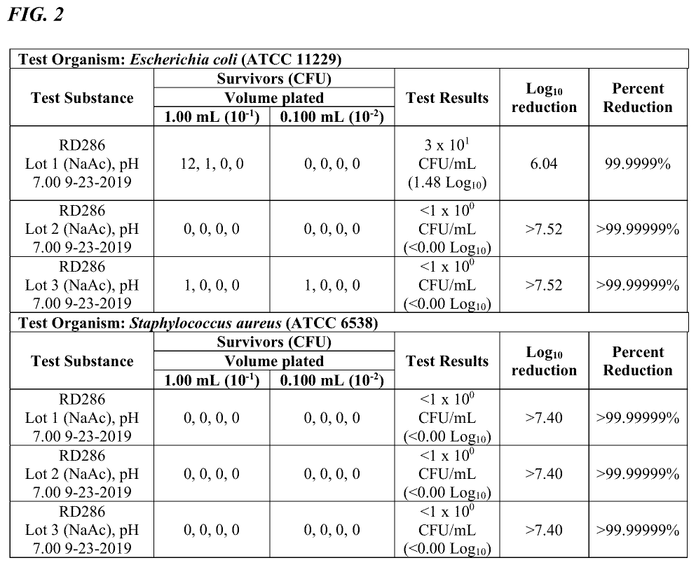

All three lots also demonstrated a >99.99999% (>7.40 Log 10) reduction of Staphylococcus aureus (ATCC 6538) following a 30 second exposure time at 25±1 (25.0° C.) in the presence of a 5% fetal bovine serum organic soil load. The results are summarized in the table depicted in FIG. 2.

### Example 3: Efficacy of Antimicrobial Composition on Non-Food Contact Surfaces

The purpose of this study was to determine the antimicrobial efficacy of spray application of a composition of Formula 1 on hard, inanimate, non-porous, non-food contact surfaces. The study was performed in compliance with the U.S. Environmental Protection Agency (EPA) requirements.

A solution of 1:15 defined as 1 part test substance (Formula 1) plus 15 parts of 400 ppm AOAC synthetic hard water was prepared. From a stock slant no more than 5 transfers from original stock and ≤1 month old, an initial tube (10 ml) of culture broth was inoculated. This culture was termed the “initial broth suspension.” From this initial broth suspension, a minimum of three daily transfers using 1 loopful (10 μL) of culture into 10 ml of culture media was performed on consecutive days prior to use as an inoculum. The S. aureus daily transfer was incubated at 35-37° C. (36.0° C.) and the Enterobacter aerogenes daily transfer was incubated at 25-32° C. (29.0° C.), for 24±2 hours using the appropriate growth medium.

A 48-54 hour (48 hour) culture was incubated at 35-37° C. (36.0° C.) for Staphylococcus aureus and at 25-32° C. (29.0° C.) for Enterobacter aerogenes. Each culture was vortex-mixed and allowed to settle for ˜15 minutes. The upper ⅔rds of the culture was removed and transferred to a sterile vessel for use in testing. The Enterobacter aerogenes culture was diluted using sterile growth medium by combining 1.0 ml of test organism suspension with 4.0 ml of sterile growth medium. The cultures were thoroughly mixed prior to use.

A 0.10 ml aliquot of FBS was added to 1.90 ml of each prepared culture to yield a 5% fetal bovine serum organic soil load.

Sterile carriers were inoculated with 0.02 ml (20.0 μl) of culture using a calibrated pipettor spreading the inoculum to within approximately 3 mm of the edges of the carrier. The inoculated carriers were dried for 20 minutes at 35-37° C. (36.0-36.1° C.) and 40-41% relative humidity with the Petri dish lids slightly ajar and appeared visibly dry following drying. A constant humidity chamber was used in place of a desiccating chamber to ensure uniform humidification conditions and to overcome slow re-equilibration of a desiccator after opening.

Following the completion of drying, each of the five test carriers were sprayed with test substance using staggered intervals. Carriers were sprayed at a distance of 6-8 inches using 6 sprays, until thoroughly wet (6 sprays used) and were allowed to expose at room temperature (20.0° C.) and 47% relative humidity for 4 minutes. Following exposure, each carrier was transferred to 20 ml of neutralizer using identical staggered intervals. The jars were vortex-mixed for 10-15 seconds to suspend the surviving organisms.

Within 30 minutes of neutralization, duplicate 1.00 ml and 0.100 ml aliquots of the neutralized solution (10°) were plated onto the recovery agar plate medium.

The S. aureus plates were incubated at 35-37° C. (36.0° C.) for 48±4 hours (44.75 hours). The E. aerogenes plates were incubated at 25-32° C. (29.0° C.) for 48±4 hours (44.75 hours). Following incubation, the subcultures were visually enumerated.

Carrier Population Control: Three inoculated, dried control carriers were treated in a fashion similar to the test procedure by misting the carriers with sterile deionized water. Following exposure, the carriers were neutralized as in the test and mixed as in the test. Ten-fold serial dilutions were prepared and duplicate 0.100 ml aliquots of the 10-1 through 10-4 dilutions were plated onto an appropriate agar. The plates were incubated as in the test procedure and enumerated. The acceptance criterion for this control is a minimum geometric mean value of 7.5×105 CFU/carrier.

Carrier Sterility Control: Concurrent with testing, a representative, uninoculated carrier was added to the neutralizer. The vessel was mixed and 1.00 ml was plated onto appropriate agar and incubated. The acceptance criterion is a lack of growth following incubation.

Neutralizer Sterility: Concurrent with testing, a 1.00 ml aliquot of neutralizer was plated onto appropriate agar and incubated. The acceptance criterion is a lack of growth following incubation.

Culture Purity: A “streak plate for isolation” was performed on each organism culture and following incubation examined in order to confirm the presence of a pure culture. The acceptance criterion for this study control is a pure culture demonstrating colony morphology typical of the test organism.

Organic Soil Load Sterility: Concurrent with testing, the serum used for the organic soil load was cultured, incubated, and visually examined for lack of growth. The acceptance criterion for this study control is lack of growth.

Neutralization Confirmation Control: In a manner consistent with the AOAC 960.09 method, the neutralization confirmation control was performed concurrent with testing. The prepared test culture was serially diluted to target 2×10⁴-2×10⁵ CFU/ml (to target a result of 10-100 CFU plated in each control run). Multiple organism dilutions were prepared.

Test Culture Titer (TCT): A 0.100 ml aliquot of diluted test organism was added to 20.0 ml of sterile diluent and vortex mixed. The mixture was held for a minimum of 30 minutes and was then spread plated utilizing duplicate 0.100 ml and 1.00 ml aliquots using the same method used in the test. The acceptance criterion for this study control is growth.

Neutralization Confirmation Control Treatment (NCT): A sterile carrier (one per test organism dilution to be used, per test substance to be evaluated) was sprayed with the test substance as in the test. The sterile carrier was allowed to expose for the exposure time and each carrier was neutralized with 20.0 ml of neutralizer. The jar was vortex-mixed for 10-15 seconds. Within 5 minutes, a 0.100 ml aliquot of diluted test organism was added to the neutralized contents and vortex mixed. The mixture was held for a minimum of 30 minutes and was then spread plated utilizing duplicate 0.100 ml and 1.00 ml aliquots using the same method used in the test. The acceptance criterion for this study control is growth within 1 log₁₀ of the test culture titer (TCT) for at least one of the aliquots plated.

Neutralizer Toxicity Treatment (NTT): A 0.100 ml aliquot of diluted test organism was added to 20.0 ml of sterile neutralizer and was vortex mixed. The mixture was held for a minimum of 30 minutes and was then spread plated duplicate 0.100 ml and 1.00 ml aliquots using the same method used in the test. The acceptance criterion for this study control is growth within 1 log₁₀ of the test culture titer (TCT) for at least one of the aliquots plated.

Inoculum Count: Each test organism was serially diluted and 0.100 ml aliquots of appropriate dilutions were plated in duplicate. The plates were incubated as in the test. This control is for informational purposes and therefore has no acceptance criterion.

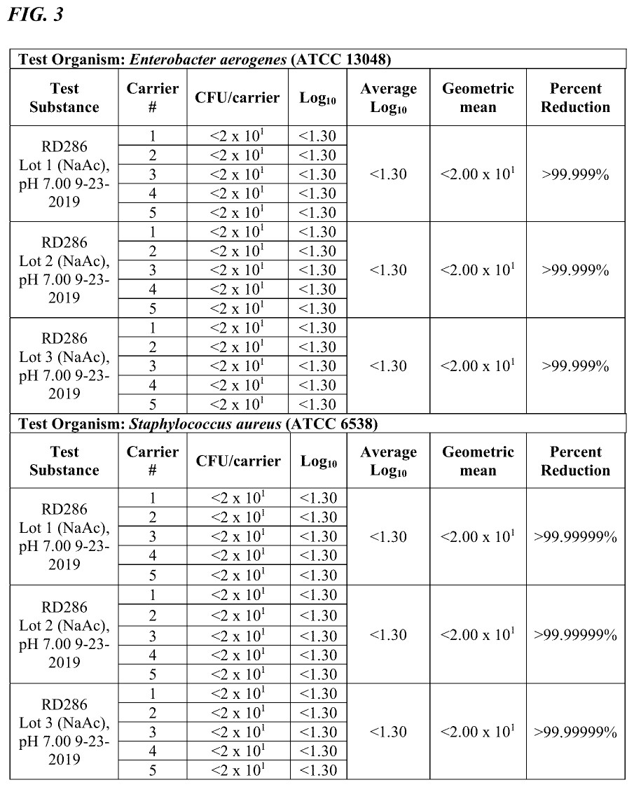

All three lots of Formula 1 as tested demonstrated a >99.999% reduction of E. aerogenes (ATCC 13048) following a 4 minute exposure time in the presence of a 5% fetal bovine serum organic soil load when tested at room temperature (20.0° C.). All three lots of Formula 1 as tested also demonstrated a >99.999% reduction of S. aureus (ATCC 6538) following a 4 minute exposure time in the presence of a 5% fetal bovine serum organic soil load when tested at room temperature (20.0° C.). These results are summarized in the Table depicted in FIG. 3.

### Example 4

Preparation of Test Organism(s): A loopful of stock slant culture was transferred to an initial 10 mL tube of growth medium. The tube was mixed and the initial culture was incubated for 24±2 hours at 35-37° C. (36.0° C.). Following incubation, a 10 pL aliquot of culture was transferred to a 20×150 mm Morton Closure tube containing 10 mL of culture medium (daily transfer #1). One additional daily transfer was prepared. The final test culture was incubated for 48-54 hours (48 hours) at 35-37° C. (36.0° C.). The test culture was vortex mixed for 3 to 4 seconds and allowed to stand for >10 minutes prior to use. After this time, the upper portion of the culture was removed, leaving behind any clumps or debris and was pooled in a sterile vessel and mixed. The culture was adjusted using sterile growth medium to target a spectrophotometer absorbance reading to (0.144) al 620 nm. The culture was then diluted using sterile growth medium by combining 1.00 mL of test organism suspension with 7.0 mL of sterile growth medium. The final test culture was mixed thoroughly prior to use.

Addition of Organic Soil Load: A 0.10 mL aliquot of FBS was added to 1.90 mL of prepared culture to yield a 5% fetal bovine serum organic soil load.

Contamination of Carriers: Individual glass slide carriers were each inoculated with 10.0 μL of culture using a calibrated pipettor. The inoculum was uniformly spread over the test surface (approximately 1 square inch) of the slide contained in the Petri dish. The dish was covered immediately and the procedure repeated until all slides were individually inoculated. The culture was vortex mixed periodically during inoculation as necessary. The carriers were allowed to dry for 30 minute at 35-37° C. (36.0-36.1° C.) and at a 50.9-55.0% relative humidity and appeared visibly dry following drying. Carriers were used in the test procedure within 2 hours of drying.

Exposure Conditions: For each lot of test substance, test carriers were sprayed, in an undisturbed horizontal position, at staggered intervals with the test substance at a distance of 6-8 inches using 6-8 sprays, until thoroughly wet (8 sprays used). The carrier was sprayed with the test substance within ±5 seconds of the exposure time following a calibrated timer.

Test System Recovery: Following the spray treatment, each treated carrier was held at room temperature (20° C.) and 50% relative humidity for 1 minute. At the end of the exposure time, the excess liquid was drained off the carrier without touching the carrier to the Petri dish or filter paper. Each treated carrier was then transferred using sterile forceps and following identical staggered intervals to 20 mL aliquots of neutralizing subculture medium. The vessel was shaken thoroughly.

Incubation and Observation: All subcultures were incubated for 48×2 hours (46 hours) at 35-37° C. (36.0° C.). Following incubation, the subcultures were visually examined for the presence or absence of visible growth.

Study Controls

Purity Gontrol: A “streak plate for isolation” was performed on the organism culture and following incubation

examined in order to confirm the presence of a pure culture. The acceptance criterion for this study control is a pure culture demonstrating colony morphology typical of the test organism.

Organic Soil Sterility Control: Concurrent with testing, the serum used for the organic soil load was cultured, incubated, and visually examined for lack of growth. The acceptance criterion for this study control is lack of growth.

Carrier Sterility Control: Concurrent with testing, a representative uninoculated carrier was added to an appropriate subculture medium. The subculture medium containing the carrier was incubated and examined for growth. The acceptance criterion for this study control is lack of growth.

Neutralizing Subculture Medium Sterility Control: Concurrent with testing, a representative sample of uninoculated neutralizing subculture medium was incubated and visually examined. The acceptance criterion for this study control is lack of growth.

Viability Control: One representative inoculated carrier was added to a vessel containing subculture medium. The vessel containing the carrier was incubated and visually examined for growth. The acceptance criterion for this study control is growth in the subculture medium.

Neutralization Confirmation Control: The neutralization of the test substance was confirmed concurrent with testing by exposing at least one sterile carrier to the test substance and transferring the carrier to subcultures containing 20 mL of neutralizing subculture medium as in the test. The subcultures were inoculated with a target of 10-100 colony forming units (CFU) of test organism, incubated under test conditions and visually examined for the presence of growth. This control was performed with multiple replicates using different dilutions of the test organism. A standardized spread plate procedure was run concurrently in order to enumerate the number of CFU actually added per tube. The acceptance criterion for this study control is growth in the subculture broth, following inoculation with <100 CFU per tube.

Carrier Population Control

Two sets of three inoculated carriers (one set prior to testing and one set following treatment) were assayed. Each inoculated carrier was individually subcultured into a vessel containing 20 mL of neutralizing subculture medium and immediately vortex mixed for 120±5 seconds. Following mixing, the contents of the three subcultured carriers were pooled (60 mL). Appropriate serial ten-fold dilutions were prepared and the duplicate aliquots were spread plated onto an agar plate medium, and incubated. Following incubation, the resulting colonies were enumerated. The individual CFU per carrier set results were calculated, and the Log₁₀ value of each carrier set determined. The average Log₁₀ value was calculated. The acceptance criterion for this study control is an average of 4.0 to 5.0 Log 10 CFU/carrier.

STUDY ACCEPTANCE CRITERIA: Test Substance Performance Criteria: The efficacy performance requirements for label claims state that the test substance must kill the microorganism on 10 out of the 10 inoculated carriers.

Control Acceptance Criteria: The study controls must perform according to the criteria detailed in the study controls description section.

Data Analysis

All data measurements including the culture purity, viability, organic soil load sterility, neutralizing subculture medium sterility, carrier sterility, carrier population and neutralization confirmation controls were within acceptance criteria.

Analysis

RD286 (Lot 05-14-20-Lab1), ready to use, demonstrated no growth of Acinetobacter baumannii(ATCC BAA-1709) in any of the 10 subcultures following a 1 minute exposure time at room temperature (20° C.) and 50% relative humidity in the presence of a 5% fetal bovine serum organic soil load.

RD286 (Lot 05-14-20-Lab3), ready to use, demonstrated no growth of Acinetobacter baumannii(ATCC BAA-1709) in any of the 10 subcultures following a 1 minute exposure time at room temperature (20° C.) and 50% relative humidity in the presence of a 5% fetal bovine serum organic soil load. (FIG. 4).

### Example 5: Effectiveness Against Additional Bacteria

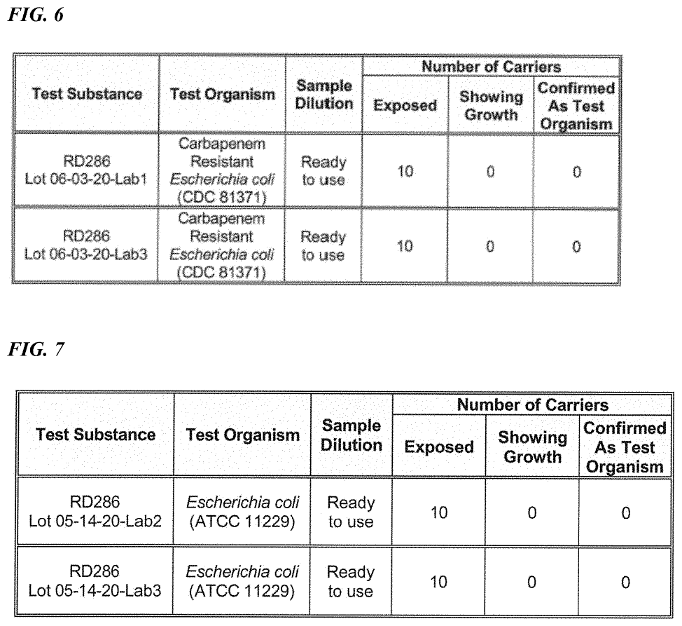

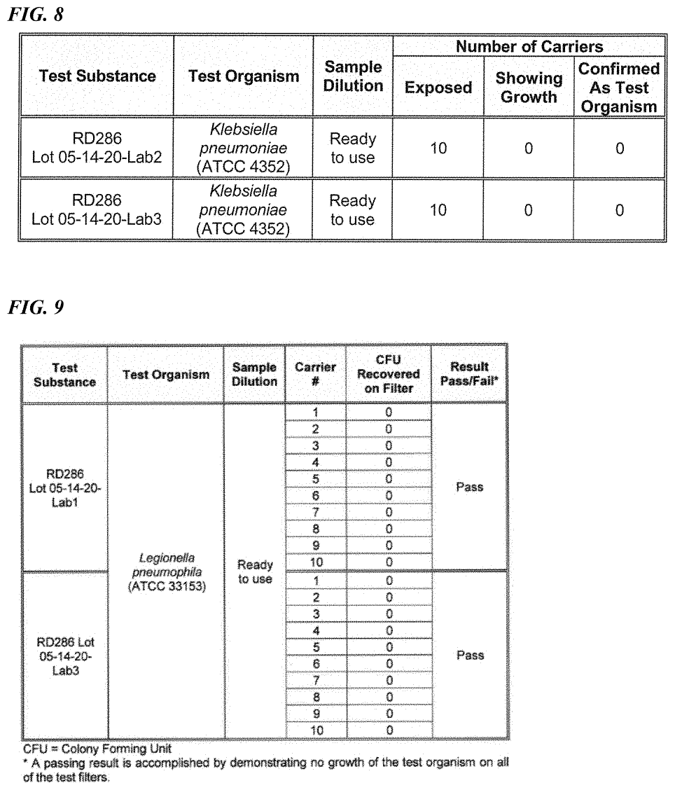

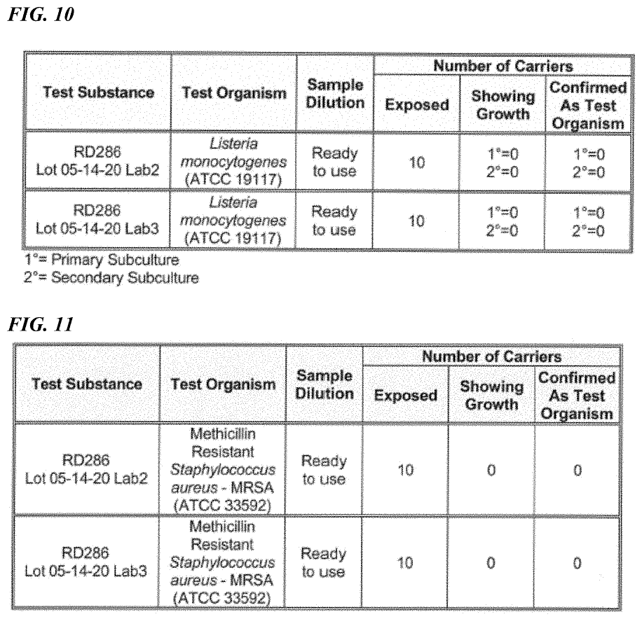

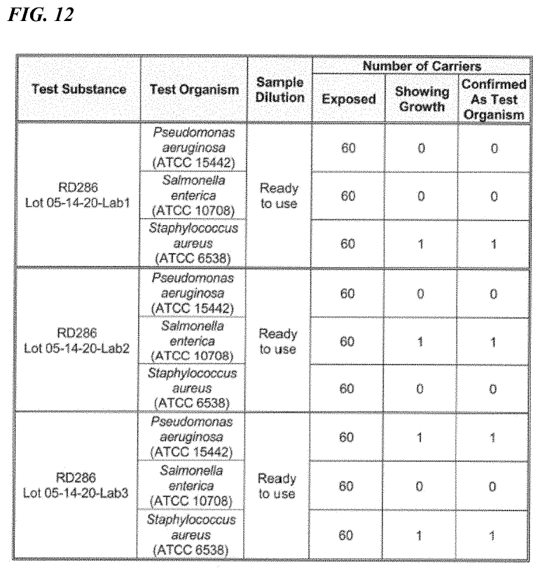

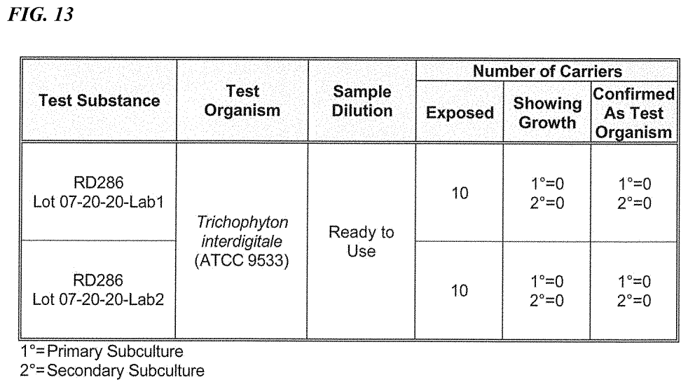

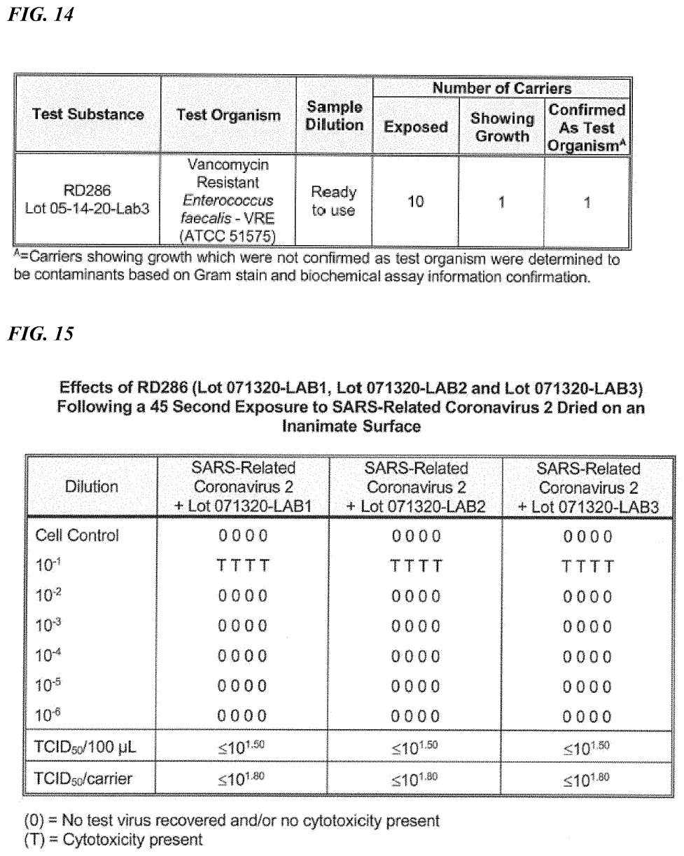

According to similar procedures as Example 4, RD286 was tested against B. pertussis (FIG. 5), E. coli FIG. 7), carbapenem-resistant E. coli (FIG. 6), K. pneumonia (FIG. 8), Legionella. pneumophila (FIG. 9), Listeria monocytogenes (FIG. 10), methicillin resistant S. aureus (FIG. 11), P. aeruginosa (FIG. 12), Salmonella enterica (FIG. 12), S. aureus (FIG. 12), Trichophyton interdigitale (FIG. 13), vancomycin-resistant Enterococcus faeclis (FIG. 14). RD286 demonstrated effectiveness against all strains of bacteria tested.

### Example 6: Effectiveness Against SARS-Related Coronavirus 2

Test System

Virus: The Isolate USA-WA1/2020 strain of SARS-Related Coronavirus 2 used for this study was deposited by the Centers for Disease Control and Prevention and obtained through BEI Resources, NIAID, NIH (BEI Resources NR-52281). The stock virus was prepared by collecting the supernatant culture fluid from 75-100% infected culture cells. The cells were disrupted and cell debris removed by centrifugation at approximately 2000 RPM for five minutes at approximately 4° C. The supernatant was removed, aliquoted, and the high titer stock virus was stored at ≤70° C. until the day of use. On the day of use, an aliquot of stock virus (Accuratus Lab Services Lot SARS2-2) was removed, thawed and maintained at a refrigerated temperature until used in the assay. The stock virus culture was adjusted to contain 5% fetal bovine serum as the organic soil load. The stock virus tested demonstrated cytopathic effects (CPE) typical of Coronavirus on Vero E6 cells. Indicator Cell CulturesCultures of Vero E6 cells were originally obtained from the American Type Culture Collection, Manassas, Va. (ATCC CRL-1586). The cells were propagated by Analytical Lab Group-Midwest personnel. The cells were seeded into multiwell cell culture plates and maintained at 36-38° C. in a humidified atmosphere of 5-7% CO₂. On the day of testing, the cells were observed as having proper cell integrity and confluency, and therefore, were acceptable for use in this study.

Test Medium: The test medium used in this study was Minimum Essential Medium (MEM) supplemented with 2% (v/v) heat-inactivated fetal bovine serum (FBS), 10 pg/mL gentamicin, 100 units/mL penicillin, 2.5 pg/mL amphotericin B, 2.0 mM L-glutamine, 0.1 mM NEAA and 1.0 mM sodium pyruvate.

Test Method

Preparation of Test Substance: Three lots of RD286 were used as received from the Sponsor and was applied using Analytical Lab Group provided trigger spray bottles. The test substance was homogeneous and was at the exposure temperature prior to use.

Preparation of Virus Films: Films of virus were prepared by spreading 200 μL of virus inoculum uniformly over the bottoms of four separate 100×15 mm sterile glass petri dishes (without touching the sides of the petri dish). The virus films were dried at 20.06° C. in a relative humidity of 48.69% until visibly dry (20 minutes).

Preparation of Sephadex Gel Filtration Columns: To reduce the cytotoxic level of the virus-test substance mixture prior to assay of virus, and/or to reduce the virucidal level of the test substance, virus was separated from the test substance by filtration through Sephadex LH-20 gel. On the day of testing, Sephadex columns were prepared by centrifuging the prepared Sephadex gel in sterile syringes for three minutes to clear the void volume. The columns were then ready to be used in the assay.

Input Virus Control (TABLE 1): On the day of testing, the stock virus utilized in the assay was titered by 40-fold serial dilution and assayed for infectivity to determine the starting titer of the virus. For each lot of test substance, one dried virus film was individually exposed for 45 seconds at room temperature (20.06° C.) and 48.69% relative humidity to the amount of spray released under use conditions. The carriers were sprayed using 6 sprays, until thoroughly wet, at a distance of 6 to 8 inches, and held covered for the exposure time. The virus films were completely covered with the test substance. Just prior to the end of the exposure time, the plates were individually scraped with a cell scraper to resuspend the contents and at the end of the exposure time the virus-test substance mixtures were immediately passed through individual Sephadex columns utilizing the syringe plungers in order to detoxify the mixtures. The filtrates (10⁻¹ dilution) were then titered by 10-fold serial dilution and assayed for infectivity and/or cytotoxicity. To aid in the removal of the cytotoxic effects to the cell cultures the 10⁻² dilutions were passed through an individual Sephadex column utilizing the syringe plungers following titration.

Treatment of Dried Virus Control Film (TABLE 4): One virus film was prepared as previously described (paragraph 2). The virus control film was exposed to 2.00 mL of test medium in lieu of the test substance and held covered for 45 seconds at room temperature (20.06° C.) and 48.69% relative humidity. Just prior to the end of the exposure time, the virus control was scraped with a cell scraper and at the end of the exposure time the virus mixture was immediately passed through a Sephadex column in the same manner as the test virus (paragraph 5). The filtrate (10⁻¹ dilution) was then titered by 10-fold serial dilution and assayed for infectivity. To mimic the test the 40-2 dilution was passed Cytotoxicity Controls (TABLE 3)

Each lot of the test substance was sprayed as previously described onto separate sterile petri dishes and held covered for the 45 second exposure time at room temperature (20.06° C.) and 48.69% relative humidity. Just prior to the end of the exposure time, the plates were individually scraped with a cell scraper and at the end of the exposure time the contents were immediately passed through a Sephadex column utilizing a syringe plunger. The filtrate (10⁻¹ dilution) was then titered by 10-fold serial dilution and assayed for cytotoxicity. Cytotoxicity of the Vero E6 cell cultures was scored at the same time as the virus-test substance and virus control cultures. To aid in the removal of the cytotoxic effects to the cell cultures the 10⁻² dilutions were passed through an individual Sephadex column utilizing the syringe plungers following titration.

Assay of Non-Virucidal Level of Test Substance (Neutralization Control): Each dilution of the neutralized test substance (cytotoxicity control dilutions) was challenged with an aliquot of low titer stock virus to determine the dilution(s) of test substance at which virucidal activity, if any, was retained. Dilutions that showed virucidal activity were not considered in determining reduction of the virus by the test substance.

Using the cytotoxicity control dilutions prepared above, an additional set of indicator cell cultures was inoculated with a 100 μL aliquot of each dilution in quadruplicate. A 100 μL aliquot of low titer stock virus (approximately 100 infectious units) was inoculated into each cell culture well and the indicator cell cultures were incubated along with the test and virus control plates.

Infectivity Assays: The Vero E6 cell line, which exhibits cytopathic effect (CPE) in the presence of SARS-Related Coronavirus 2, was used as the indicator cell line in the infectivity assays. Cells in multiwell culture dishes were inoculated in quadruplicate with 100 μL of the dilutions prepared from test and control groups. The input virus control was inoculated in duplicate. Uninfected indicator cell cultures (cell controls) were inoculated with test medium alone. The cultures were incubated at 36-38° C. (37.0° C.) in a humidified atmosphere of 5-7% CO2 (6.0% CO2) in sterile disposable cell culture labware. The cultures were scored periodically for seven days for the absence or presence of CPE, cytotoxicity, and for viability.

Calculation of Titers: Viral and cytotoxicity titers will be expressed as −log₁₀ of the 50 percent titration endpoint for infectivity (TCID₅₀ or cytotoxicity (TCD₅₀), respectively, as calculated by the method of Spearman Karber.

Per Volume Inoculated (TCID₅₀/volume inoculated):

$$\text{-} Log of 1 st dilution inoculated - [ ( ( \frac{Sum of \% mortality at each dilution}{100} ) - 0.5 ) \times ( logarithm of dilution ) ]$$

### Per Carrier (TCID₅₀/carrier):

(Antilog of TCID₅₀*X(volume inoculated per carrier/volume inoculated per well)=Y

Log of Y=the TCID₅₀/carrier(Example: 10^{5.50} or 5.50 Log₁₀)

*(TCD₅₀ value calculated based on the volume inoculated per well.

Calculation of Log Reduction: The following calculation was used to calculate the log reduction per volume inoculated per well and the log reduction per carrier.

Dried Virus Control Log₁₀ TCID₅₀−Test Substance Log₁₀ TCID₅₀=Log Reduction

Calculation of Infections

### Units

$$( \frac{input virus titer}{dilution of test virus for neutralization control} ) ( \frac{low titer virus inoculation volume}{input virus inoculation volume} ) = \text{\textasciitilde} infectious units$$

Example: Titer of the input virus: 10^{5.50} (TCID₅₀ of 10^{6.00}), 1:1,000 dilution made from stock virus for use in the neutralization control, 100 4 L/well of low titer virus inoculated and 250 μL/well of input virus inoculated)

(10^{5.50}/10^{3.00})(100 μL/250 μL)=˜126 infectious units

Study Results: Results of tests with three lots of RD286 (Lot 071320-LAB1, Lot 071320-LAB2 and Lot 071 320-LAB3), ready to use and applied as a trigger spray, exposed to SARS-Related Coronavirus 2 in the presence of a 5% fetal bovine serum organic soil load at room temperature (20.06° C.) and 48.69% relative humidity for 45 seconds is shown in FIG. 15. All cell controls were negative for test virus infectivity.

The titer of the input virus control was 5.50 log₁₀/100 μL. The titer of the dried virus control was 5.50 log₁₀/100 μL (5.80 log₁₀/carrier). Following exposure, test virus infectivity was not detected in the virus-test substance mixture in any lot at any dilution tested

[≤1.50 log₁₀/100 μL (<1.80 log₁₀/carrier)]. Test substance cytotoxicity was observed in all lots at 1.50 log₁₀/100 μL. The neutralization control (non-virucidal level of the test substance) indicates that the test substance was neutralized at ≤1.50 log₁₀/100 μL for all lots.

Taking the cytotoxicity and neutralization control results into consideration, the reduction in viral titer, per volume inoculated per well and per carrier was ≥4.00 log₁₀, for all lots.

### Example 7

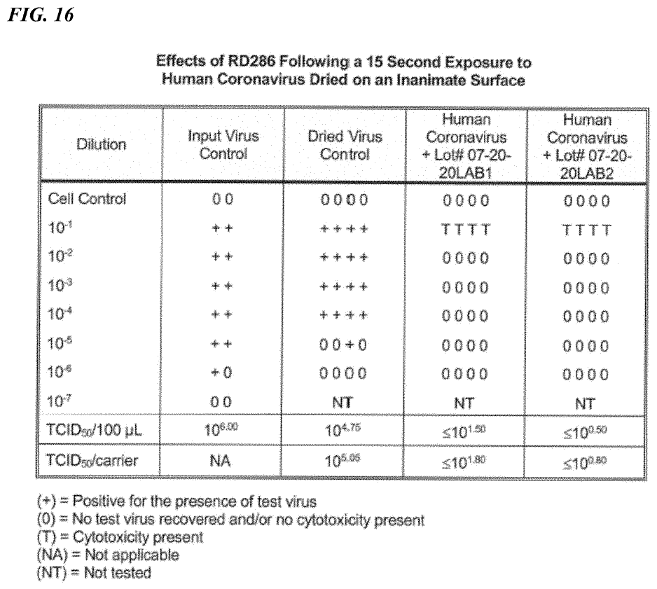

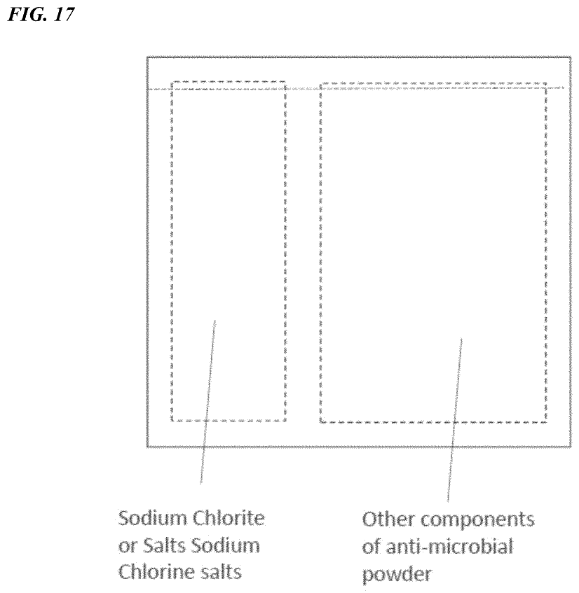

In a procedure similar to Example 6, RD286 was tested for anti-viral against human coronavirus (ATCC VR-740). Following exposure, test virus infectivity was not detected in the virus-test substance mixture for either lot at any dilution tested [≤1.50 log 10/100 μL (5.05 log 10/carrier)]. Test substance cytotoxicity was observed in both lots at 1.50 log 10/100 μL. The neutralization control (non-virucidal level of the test substance) indicates that the test substance was neutralized at ≤1.50 log 10/100 μL for both lots (FIG. 16). Taking the cytotoxicity and neutralization control results into consideration, the reduction in viral titer, per volume inoculated per well and per carrier was >3.25 log 10 for both lots.

### Example 8: Comparative Minimal Inhibitory Concentration (MIC) of Sodium Chlorite, Stepan BTC 2125M, RD286 and RD290

The effectiveness of Sodium Chlorite, Stepan BTC 2125M, RD286 and RD290 were evaluated by determining their minimal inhibitory concentrations against Pseudomonas aeruginosa ATCC 27853. Pseudomonas aeruginosa was chosen for the testing because Pseudomonas aeruginosa is the hardest to kill amongst the bacteria that are problematic pathogens and pose risks to human health.

TABLE 2

| | *MIC against Pseudomonas |
|---|---|
| Antibacterial | aeruginosa ATCC 27853 |
| 0.5% Sodium Chlorite | ⅛ |
| 0.5% Stepan BTC 2125M | 1/32 |
| RD286 | 1/64 |
| RD290 | 1/128 |

*The MIC is the greatest dilution at which 100% of a starting culture of 1,000,000 or 1 × 10⁶ bacteria are killed

The data shows that the RD286 and RD290 formulations were more effective at killing Pseudomonas aeruginosa ATCC 27853 than Sodium Chlorite and Stepan BTC 2125M individually, which are the active ingredients in those formulations.

Protocol for determining the minimal inhibitory concentration (MIC) of antimicrobials

This protocol has been designed to accurately determine the killing of microorganisms by antimicrobial agents, since this is the guarantee most often used in preventative products, such as disinfectants and sanitizers, for legal purposes. In a standard MIC assay a growing culture of 1,000,000 or 10⁶ microorganisms is subjected to serial 1:2 (½) dilutions of the preventative agent and then their growth is observed 16 to 24 hours later. A 99.9% killing is observed if only 1000 or 10³ microorganisms remain. A 99.99% killing is observed if only 100 or 10² microorganisms remain.

Day one: Prepare 2 mL overnights in 1× media, for example, 1× tryptic soy broth (TSB), of the microorganisms to be tested.

Day two: The ½ dilution of the antimicrobial to be tested is prepared by diluting 5 mL of the original antimicrobial solution to be diluted with 5 mL of dH₂O and thoroughly mixing the resulting solution. The ¼ dilution is prepared by diluting 5 mL of the ½ diluted solution with 5 mL of dH₂O and thoroughly mixing the resulting solution. ⅛, 1/16, 1/32, 1/64, 1/128, 1/256 and 1/512 dilutions are also prepared.

A saturated 1×TSB overnight of the microorganism to be tested is diluted to 2,000,000 or 2×10⁶ colony forming units per mL in 2×TSB. Pseudomonas aeruginosa strains typically yield saturated overnights that contain about 1×10¹⁰ CFU per mL.

1 mL of the diluted overnight is added to 1 mL of each of the dilutions of the antimicrobial agent to be tested, ranging from ½ to 1/1024, in culture tubes. Notes . . . The ½ dilution of the antimicrobial agent yields a final ¼ dilution, etc. . . . . The final concentration of bacteria that is tested is 1,000,0000 or 1×10⁶ CFU.

The cultures are then aerated at the appropriate temperature for 16 to 24 hours.

Day three: The tubes are checked for growth to determine what are the highest dilutions of antimicrobial agent that prevents the growth of the microorganisms. These are noted and recorded. The dilution that does not inhibit growth and the next two lower dilutions that inhibit growth are also noted and recorded.

The greatest dilution that yields complete killing of the microorganism and the next two lower dilutions are further evaluated by plating 100 ul of the overnight solution onto the appropriate plate, for example 1×TSB plates.

Once the spread media has dried, the plates are incubated at the proper temperature overnight

Day four: Whether dilutions that appeared not to have any growth in broth actually prevented growth are noted. Only a dilution that showed no growth in broth and yielded no colonies when plated is a dilution that truly prevented microbial growth.

Although there have been described particular embodiments of the present invention of a new and useful ANTIMICROBIAL COMPOSITION, it is not intended that such references be construed as limitations upon the scope of this invention.

1. An antimicrobial composition comprising: a water solution comprising a chlorite salt having a concentration ranging from about 2,000 parts per million to about 8,000 parts per million, and at least one quaternary ammonium salt having a concentration ranging from about 5,000 parts per million to about 10,000 parts per million.

2. The antimicrobial composition of claim 1, wherein: the concentration of the chlorite salt to the water solution ranges from about 5,000 parts per million to about 8,000 parts per million, and the at least one quaternary ammonium salt has a concentration ranging from about 6,000 parts per million to about 10,000 parts per million.

3. The antimicrobial composition of claim 1 or 2, wherein the quaternary ammonium salt comprises an n-alkyl dimethyl benzyl ammonium chloride, an n-alkyl dimethyl ethylbenzyl ammonium chloride, didecyldimethylammonium chloride, didecyldimethylammonium bromide, cetalkonium chloride, cetalkonium bromide, cetylpyridinium chloride, cetylpyridinium bromide, cetyltrimethylammonium bromide, cetyltrimethylammonium chloride, cetrimonium, tetraethylammonium bromide, domiphen bromide, domiphen chloride, dofanium chloride, benzethonium chloride, benzyl(C₁₂₋₁₈)alkyldimethylammonium chloride, benzyldodecyldimethylammonium bromide, benzyldodecyldimethylammonium chloride, dodecyltrimethylammonium bromide, dodecyltrimethylammonium chloride, hexadecyltrimethylammonium bromide, hexadecyltrimethylammonium chloride, methylbenzethonium chloride, tetradecyltrimethylammonium bromide, tetradecyltrimethylammonium chloride, tetraethylammonium bromide, tetraethylammonium chloride, or any combination thereof.

4. The antimicrobial composition of claim 3, wherein the quaternary ammonium salt comprises an n-alkyl dimethyl benzyl ammonium chloride and an n-alkyl dimethyl ethyl benzyl ammonium chloride.

5. The antimicrobial composition of claim 4, wherein the alkyl group on the n-alkyl dimethyl benzyl ammonium chloride comprises C₁₂, C₁₄, C₁₆ and C₁₈ carbon groups.

6. The antimicrobial composition of claim 4, wherein the alkyl group on the n-alkyl dimethyl ethylbenzyl ammonium chloride comprises C₁₂ and C₁₄ carbon groups.

7. The antimicrobial composition of claim 4, wherein the n-alkyl dimethyl benzyl ammonium chloride comprises about 5% C₁₂, about 60% C₁₄, about 30% C₁₆, and about 5% C₁₈ carbon groups, and the n-alkyl dimethyl ethylbenzyl ammonium chloride comprises about 68% C₁₂ and about 32% C₁₄ carbon groups.

8. The antimicrobial composition of any one of claims 1 to 7, further comprising sodium tetraborate in a concentration ranging from about 8,000 parts per million to about 15,000 parts per million.

9. The antimicrobial composition of any one of claims 1 to 8, further comprising a buffer.

10. The antimicrobial composition of claim 9, wherein the buffer comprises sodium bicarbonate, ferric chloride, citric acid, sodium percarbonate, trisodium phosphate, acetic acid, sodium acetate, or any combination thereof.

11. The antimicrobial composition of claim 10, wherein the buffer comprises the sodium acetate in a concentration ranging from about 500 to about 1500 parts per million.

12. The antimicrobial composition of claim 10, wherein the buffer further comprises acetic acid in a concentration ranging from about 100 to about 5000 parts per million, where the acetic acid has a dilution ratio of about 1:8 to about 1:12,

13. The antimicrobial composition of any one of claims 1 to 12, further comprising a surfactant having a concentration ranging from about 100 parts per million to about 3,000 parts per million.

14. The antimicrobial composition of claim 13, wherein the surfactant comprises a non-ionic surfactant.

15. The antimicrobial composition of claim 13, wherein the surfactant comprises an alkoxylated non-ionic surfactant.

16. The antimicrobial composition of claim 15, wherein the alkoxylated non-ionic surfactant comprises ethoxylated alcohols.

17. The antimicrobial composition of claim 16, wherein the ethoxylated alcohols are C₉-C₁₁ ethoxylated alcohols.

18. The antimicrobial composition of any one of claims 1 to 17, wherein the pH of the composition ranges from about 6.8 to about 7.2.

19. The antimicrobial composition of any one of claims 1 to 18, wherein the antimicrobial composition has a sporicidal efficacy of substantially 100 percent against endospores of Clostridium difficile ATCC 43598 in an ASTM E2315 compliant test after a contact time up to about 120 seconds.

20. The antimicrobial composition of any one of claims 1 to 19, wherein the antimicrobial composition has a sporicidal efficacy of substantially 100 percent against endospores of Escherichia Coli in an ASTM E2315 compliant test after a contact time of about 30 seconds.

21. An antimicrobial composition comprising water solution comprising:
about 5,000 parts per million of a chlorite salt,
about 7,000 parts per million of a quaternary ammonium compound, and
about 100 parts per million of an ethoxylated alcohol surfactant.

22. The antimicrobial composition of claim 21, wherein the quaternary ammonium compound comprises an n-alkyl dimethyl benzyl ammonium chloride and an n-alkyl dimethyl ethyl benzyl ammonium chloride.

23. The antimicrobial composition of claim 21 or 22, further comprising about 10,000 parts per million of sodium tetraborate.

24. The antimicrobial composition of any one of claims 21 to 23, further comprising about 800 parts per million of sodium acetate and about 3200 parts per million of acetic acid, wherein the acetic acid has a 1:10 dilution.

25. The antimicrobial composition any one of claims 21 to 24, wherein the composition further comprises sodium acetate, ferric chloride, citric acid, sodium percarbonate, trisodium phosphate, or any combination thereof in a concentration ranging from about 500 to about 1500 parts per million.

26. A method for disinfecting an object comprising applying the composition of any one of claims 1 to 25 to the object.

27. The method of claim 26, wherein the object is a hard surface or a soft surface.

28. The method of claim 26 or 27, wherein the object is contaminated with a bacteria or a virus, and the method kills at least 99.5% of the virus or bacteria on the object.

29. The method of claim 28, wherein the bacteria comprises Clostridium difficile, Staphylococcus aureus, Escherichia coli, Pseudomonas aeruginosa, Enterobacter aerogenes, Acinetobacter baumanni, B. pertussis, E. coli, carbapenem-resistant E. coli, K pneumonia, Legionella. pneumophila, Listeria monocytogenes, methicillin resistant S. aureus, P. aeruginosa, Salmonella enterica, S. aureus, Trichophyton interdigitale, vancomycin-resistant Enterococcus faeclis.

30. The method of any one of claims 26 to 28, wherein the virus comprises COVID-19, SARS, MERS, influenza, or any combination thereof.

31. A method of disinfecting an agricultural product comprising applying the composition of any one of claims 1 to 25 to the agricultural product.

32. The method of claim 31, wherein the method substantially kills fungus, mold, spores, bacteria, or viruses on the agricultural product.

33. The method of claim 31 or 32, wherein the agricultural product in a cannabis plant.

34. An antimicrobial hand care composition comprising sodium chlorite in an amount ranging from about 0.4-0.5% by weight, a quaternary ammonium salt in an amount ranging from about 0.6 to about 0.7% by weight, a surfactant in an amount ranging from about 0.05% to about 0.1% by weight, sodium tetraborate in an amount ranging from about 0.5% to about 1.0% by weight, an emollient compound in an amount ranging from about 0.1 to about 0.5% by weight, and up to about 97.5% by weight of deionized water.

35. An antimicrobial hand care composition comprising sodium chlorite in an amount ranging from about 0.4-0.5% by weight, a quaternary ammonium salt in an amount ranging from about 0.6 to about 0.7% by weight, a surfactant in an amount ranging from about 0.05% to about 0.1% by weight, baking soda (sodium bicarbonate, NaHCO₃), iron chloride (FeCl₃), citric acid (C₆H₈O₇), sodium percarbonate(Na₂H₃CO₆), trisodium phosphate (Na₃PO₄) in an amount ranging from about 0.07% to about 0.88% by weight %, an emollient compound in an amount ranging from about 0.1 to about 0.5% by weight, and up to about 97.5% by weight of deionized water.

36. The antimicrobial hand care composition of claim 34 or 35, wherein the emollient compound comprises glycerin, shea butter, cocoa butter, lanolin, propylene glycol, or any combination thereof.

37. The antimicrobial hand care formulation of any one of claims 34 to 36, wherein the quaternary ammonium compound comprises the quaternary ammonium salt comprises an n-alkyl dimethyl benzyl ammonium chloride, an n-alkyl dimethyl ethylbenzyl ammonium chloride, didecyldimethylammonium chloride, didecyldimethylammonium bromide, cetalkonium chloride, cetalkonium bromide, cetylpyridinium chloride, cetylpyridinium bromide, cetyltrimethylammonium bromide, cetyltrimethylammonium chloride, cetrimonium, tetraethylammonium bromide, domiphen bromide, domiphen chloride, dofanium chloride, benzethonium chloride, benzyl (C₁₂₋₁₈)alkyldimethylammonium chloride, benzyldodecyldimethylammonium bromide, benzyldodecyldimethylammonium chloride, dodecyltrimethylammonium bromide, dodecyltrimethylammonium chloride, hexadecyltrimethylammonium bromide, hexadecyltrimethylammonium chloride, methylbenzethonium chloride, tetradecyltrimethylammonium bromide, tetradecyltrimethylammonium chloride, tetraethylammonium bromide, tetraethylammonium chloride, or any combination thereof.
